(19) United States
(12) Patent Application Publication
(10) Pub. No.: US 2025/0076275 A1
(43) Pub. Date: Mar. 6, 2025

(54) WHOLE LIFECYCLE MULTI-INDICATOR SYNCHRONOUS DETECTION DEVICE AND METHOD FOR YARNS OR FABRICS

(71) Applicant: Wuhan Textile University, Wuhan (CN)

(72) Inventors: Zhigang Xia, Wuhan (CN); Jun Wu, Wuhan (CN); Weimin Cao, Wuhan (CN); Jiandong Tang, Wuhan (CN); Fei Li, Wuhan (CN); Bin Li, Wuhan (CN); Weilin Xu, Wuhan (CN)

(21) Appl. No.: 18/951,640

(22) Filed: Nov. 18, 2024

Related U.S. Application Data
(63) Continuation of application No. PCT/CN2023/111054, filed on Aug. 3, 2023.

(30) Foreign Application Priority Data
Aug. 15, 2022 (CN) 202210973744.6
Nov. 11, 2022 (CN) 202211415279.0

(51) Int. Cl. G01N 33/36; B65H 59/18; G01N 27/60

(52) U.S. Cl. CPC G01N 33/365; B65H 59/18; G01N 27/60; B65H 2701/31

(57) ABSTRACT

A device and a method of whole lifecycle multi-indicator synchronous detection of yarns or fabrics are provided. The device includes a hairiness detection unit, a first guide unit, a tension adjustment unit, and a friction unit. The device is used for circularly rubbing a closed-loop object to be detected with a preset length on the friction unit, and collecting hairiness data in real time through the hairiness detection unit. The tension adjustment unit is a free-falling tension adjustment unit used to automatically adjust tension of the object to be detected in real time to ensure the stability of friction and transmission of the object to be detected. By arranging the circulating friction closed-loop circuit and arranging the free-falling tension adjustment unit on the circuit, the friction stability is improved. The synchronous and rapid detection of multiple indicators of the object to be detected can be realized.

### CROSS-REFERENCE OF RELATED APPLICATIONS

This application is a continuation of International Application No. PCT/CN2023/111054, filed on Aug. 3, 2023. The international Application claims priority to a Chinese patent application No. CN202210973744.6, filed on Aug. 15, 2022 and a Chinese patent application No. CN202211415279.0, filed on Nov. 11, 2022. The entire contents of the above-mentioned applications are hereby incorporated by reference.

### TECHNICAL FIELD

The disclosure relates to the technical field of yarn or fabric performance detection devices, and more particularly to a multi-indicator synchronous detection method and its device for the whole lifecycle of yarns or fabrics.

### BACKGROUND

Hairiness is one of the most important indicators to measure the quality of yarns and fabrics. Yarn hairiness refers to a fiber tail or a fiber loop exposed on the surface of a staple spun yarn. Hairiness, as one of the important indicators to evaluate yarn quality, not only affects yarn quality and performance, but also has a negative impact on subsequent weaving efficiency and product performances. When the yarns are stressed, the hairiness lacks of holding stress points, so the hairiness has no contribution to the yarn strength, and the yarns with excessive hairiness are easy to be worn and broken in the subsequent processing, decreasing the processing efficiency. In the weaving process, the yarns with excessive hairiness are easily entangled together, resulting in unclear openings and decreasing the weaving efficiency and the quality of the resultant fabric.

The commonly used evaluation indicators of yarn hairiness include hairiness index of hairiness number distribution and hairiness H value. Hairiness index is to describe yarn hairiness by the number of hairiness with different set lengths (it is defined as the cumulative number of hairiness with a length exceeding a set length on a single side in a unit length of the yarn, and the unit is root/meter). Hairiness H value reflects a total amount of hairiness on a measured yarn per unit length (it is defined as a total length of fibers protruding within a measured range of 1 centimeter long of the measured yarn). The yarn quality, processing and weaving performances can all be evaluated by the hairiness evaluation indicators.

However, one kind of yarn hairiness is its own hairiness after the yarn is produced, and the other is regenerated hairiness of the yarn after subsequent repeated rubbing. When the regenerated hairiness reaches a certain number and length, serious pilling will occur, which will also lead to fabric pilling and poor smoothness. The device provided by the related art has singleness, and can only be simply used for testing yarn hairiness, or a section of yarn is repeatedly rubbed by an abrasion resistance tester to test its abrasion resistance. The own hairiness of the yarn is generally obtained by testing the total amount of hairiness on a certain length of yarn and then averaging it. For example, a Chinese Patent Application No.

CN201420606153.6 (with publication No. CN204203106U) discloses a yarn hairiness detection apparatus, including a yarn transmission device, a yarn image acquisition device and a yarn image processing device. After the yarns are unwound from a bobbin (also referred to as yarn cop), the yarns stably pass through the field of view of an area-array camera with enlarger lens under a constant tension on a yarn path formed by a yarn guide hook and a yarn guide wheel, and finally the yarns are output by a power roller driven by a driving motor. When the yarns pass through the yarn image acquisition device, the area-array camera and a stroboscopic background surface light source (also referred to as flashing backlight) are triggered synchronously, so that a background brightness of an acquired yarn image meets detection requirements, and a triggering frequency of the area-array camera is matched with a current yarn guide speed, so that the yarn image can be acquired completely and without redundancy. This method is a conventional non-closed cyclic hairiness testing method.

A Chinese Patent Application No. CN201720561765.1 (with publication No. CN206990379U) discloses a yarn wear-resistant detection device, in which two ends of the yarn are bound by a mounting seat and a counterweight, an upper roller is connected to a computer, and the number of revolutions of the upper roller is recorded by the computer. Two rollers are arranged to rub the yarn repeatedly, and then a broken state of the yarn is detected by the counterweight. Therefore, in the related art, the testing of yarn hairiness or abrasion resistance has its own testing methods and standards, and it is difficult to realize the comprehensive testing of various performance indicators of yarn, so the practicability is not good.

A Chinese Patent Application No. CN201810015923.2 (with publication No. CN108240938A) discloses an online detection method for surface hairiness of a circulating friction yarn. This method sets up a closed-loop yarn loop, and sets up a friction roller in the loop, so that the yarn repeatedly receives friction in the process of circulating transmission, and the hairiness condition is detected in real time through a hairiness collection device in the loop. Although this method can monitor the yarn friction online in real time, it still has certain defects as follows. 1. For cyclic friction, a tension yarn guide cannot realize real-time automatic adjustment, which will seriously affect the friction stability, and then affect the accuracy of the detection results, and even gradually become unable to circulate because of the continuous reduction of yarn tension. 2. The closed-loop yarn loop is provided with a bristle wheel, and a hairiness monitoring part is provided with an air suction pipe. These two settings will affect a natural state of the hairiness on the yarn, which will increase influencing factors of the hairiness and further affect the stability and accuracy of the detection. 3. Only dynamic hairiness and friction fracture properties of the yarn can be obtained, but other properties such as yarn creep cannot be obtained at the same time. 4. The length of the yarn loop is not limited, so it is not suitable for the closed-loop test of long yarn from the perspective of its device layout. When the yarn loop is too short, the randomness of the test is greater, and the accuracy of the test results is reduced.

In view of this, it is necessary to design a new multi-indicator synchronous detection method and its corresponding device for whole lifecycle of yarns or fabrics to solve the above problems.

### SUMMARY

In order to overcome shortcomings of the related art, the purpose of the disclosure is to provide a device and a method for whole lifecycle multi-indicator synchronous detection of yarns or fabrics. By setting a closed loop of circulating friction and setting a free-falling tension adjustment unit on the loop, the yarns or the fabric strips can maintain a certain tension from beginning to end in the process of friction creep, and the friction stability is improved. At the same time, the hairiness data of the yarns or the fabric strips are collected in real time until the yarns or the fabric strips are worn and broken, and the abrasion resistance, strength, evenness appearance and creep performance of the yarns can be evaluated through the dynamic hairiness data of the whole process of the yarns or the fabric strips, so that the multi-performance indicators of the functional yarns or fabric strips can be quickly detected in the whole lifecycle, and the practicability and convenience are significantly improved.

In order to achieve the purpose of the disclosure, the disclosure provides a device for whole lifecycle multi-indicator synchronous detection of a yarn or a fabric, which includes a hairiness detection unit, a first guide unit, a tension adjustment unit and a friction unit. The device is configured (i.e., structured and arranged) to make a closed-loop yarn or a closed fabric strip with a preset length on the friction unit be circularly rubbed. The hairiness detection unit is configured to collect hairiness data of the yarn or the fabric strip in real time. The tension adjustment unit is a free-falling tension adjustment unit, and is configured to automatically regulate a tension of the yarn or the fabric strip in real time to ensure stability of friction and transmission of the yarn or the fabric strip.

The disclosure further provides a method for whole lifecycle multi-indicator synchronous detection of a yarn or a fabric, which adopts the multi-indicator synchronous detection device for the whole lifecycle of the yarn or the fabric, and the method includes the following steps:
S1, sequentially passing a closed-loop yarn with a preset length or a fabric strip subjected to protective pretreatment through the hairiness detection unit, the first guide unit, the tension adjustment unit and the friction unit to form a circulating transmission loop;
S2, starting the hairiness detection unit and a transmission assembly, so as to drive the yarn or the fabric strip at a uniform speed, and acquiring a hairiness length and a quantity on the yarn or the fabric strip in real time until the yarn or the fabric strip is worn and broken; and
S3, processing all broken data to obtain a plurality of groups of performance data of the yarn or the fabric strip, so as to complete the whole lifecycle multi-indicator synchronous detection of the yarn or the fabric strip.

The disclosure has beneficial effects as follows.

1. According to the device for whole lifecycle multi-indicator synchronous detection of the yarns or the fabric provided by the disclosure, a closed-loop of circulating friction is arranged, and the free-falling tension adjustment unit is arranged on the loop, so that the yarn or the fabric strip always keeps a certain tension in the process of friction creep, and the friction stability is improved. At the same time, real-time data such as hairiness change, yarn imperfection change, length change, abrasion resistance times and so on are collected in the whole lifecycle of the yarn or the fabric strip until the yarn or the fabric strip is worn and broken, so as to comprehensively evaluate the abrasion resistance, strength, evenness appearance, dynamic creep and other properties of different materials, structures, thicknesses and functional yarns, so as to realize rapid detection of the properties of multiple functional yarns or fabric strips, and the practicability and convenience are significantly improved.

2. By adopting the free-falling tension adjustment unit and setting the displacement scale line on the slide rail, the length elongation of the yarn or the fabric strip can be calculated by the displacement value of the tension rod, and then the dynamic creep performance data of the yarn or fabric strip in the whole lifecycle until the yarn or the fabric strip is worn and broken can be obtained. It is convenient to compare and fit multiple groups of dynamic data, so as to evaluate and predict the performance of yarns or fabric strips in comprehensively. Compared with the single detection method and device in the related art, the disclosure provides a new device and detection idea for the comprehensive performance evaluation of yarns or fabric strips, and is expected to obtain a new performance evaluation standard of yarns or fabric strips, thereby improving the efficiency and accuracy of the performance detection of yarns or fabric strips.

3. When the fabric strip is detected, the double-sided adhesive tape is used for protective pretreatment in advance, so that the edges of the fabric strip are bonded to the inside of the double-sided adhesive tape, and the detection result is prevented from being unstable due to the scattering or deformation of the edges of the fabric strip through the fixing action of the double-sided adhesive tape, so that the friction data of one side of the fabric strip can be accurately detected, and the detection error can be reduced. At the same time, through the fixing function of the double-sided adhesive tape, the double-sided adhesive tape is used as the skeleton to provide certain shaping and supporting functions for the fabric strip, so that the application range of the device is wider, and the obtained data are more accurate, and the device is especially suitable for the fabric strip whose edges are easy to be scattered or deformed, such as knitted fabrics and woven fabrics, e.g., woolen fabrics and non-woven fabrics, so as to solve the technical problem that the existing hairiness testing technology and device cannot quickly and synchronously detect the knitted fabrics and non-woven fabrics.

### BRIEF DESCRIPTION OF DRAWINGS

FIG. 1 illustrates a schematic structural view of a multi-indicator synchronous detection device for the whole lifecycle of yarns or fabrics according to a first embodiment of the disclosure.

FIG. 2 illustrates a schematic structural view of a tension adjustment unit illustrated in FIG. 1.

FIG. 3 illustrates a schematic structural view of a multi-indicator synchronous detection device for the whole lifecycle of yarns or fabrics according to a second embodiment of the disclosure.

FIG. 4 illustrates a schematic cross-sectional view of the tension adjustment unit which connected with an electrometer in FIG. 1.

FIG. 5 illustrates a flowchart of protective pretreatment of fabric strips.

FIGS. 6A-6D illustrate relationship curves between the number of hairiness and the number of cycles of different yarns.

FIG. 7 illustrates relationship curves between the number of pilling and the number of cycles of different yarns.

FIG. 8 illustrates a physical picture of yarn evenness appearance of 18tex yarn under different cycle times.

FIG. 9 illustrates a physical picture of yarn evenness appearance of 24tex yarn under different cycle times.

### DESCRIPTION OF REFERENCE SIGNS

10—hairiness detection unit; 11a, 11b—hairiness acquisition module; 12a, 12b—first guide roller; 13a, 13b—second guide roller; 14a, 14b—first roller; 15a, 15b—second roller; 16a, 16b—image acquisition assembly; 20—first guide unit; 21—first support rod; 22a, 22b—third guide roller; 23—fixing plate; 30—tension adjustment unit; 31a, 31b—slide rail; 32a, 32b—tension rod; 40—friction unit; 41—second support rod; 42a, 42b—friction roller; 43a, 43b—fourth guide roller; 50a—yarn; 50b—fabric strip; 60—second guide unit; 70—double-sided adhesive tape.

### DETAILED DESCRIPTION OF EMBODIMENTS

In order to make the purpose, technical scheme and advantages of the disclosure clearer, the disclosure will be described in detail with specific embodiments.

Here, it should also be noted that, in order to avoid obscuring the disclosure with unnecessary details, only the structure and/or processing steps closely related to the scheme of the disclosure are shown in the specific embodiments, and other details not related to the disclosure are omitted.

In addition, it should be noted that the terms “including”, “containing” or any other variation thereof are intended to cover non-exclusive inclusion, so that a process, a method, an article or an equipment including a series of elements includes not only those elements, but also other elements not explicitly listed, or elements inherent to such process, method, article or equipment.

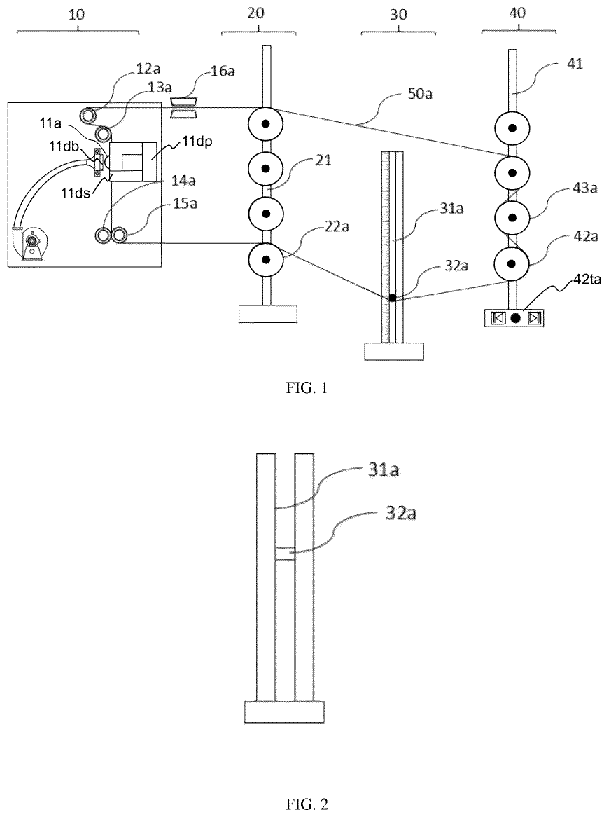

Referring to FIGS. 1 to 4, the disclosure provides a device for whole lifecycle multi-indicator synchronous detection of yarns or fabrics, which includes a hairiness detection unit 10, a first guide unit 20, a tension adjustment unit 30 and a friction unit 40. It is used to circularly rub a closed-loop yarn 50a or a fabric strip 50b with a preset length on the friction unit 40 and collect hairiness data in real time through the hairiness detection unit 10. The tension adjustment unit 30 is a free-falling tension adjustment unit 30, and the length of the yarn 50a or the fabric strip 50b will increase slowly due to creep during the friction process. Through the tension adjustment unit 30, the tension of the yarn 50a or the fabric strip 50b can be automatically adjusted in real time, so that the yarn 50a or the fabric strip 50b is always transmitted to the friction unit 40 with constant tension, so as to ensure the stability of the friction and transmission of the yarn 50a or the fabric strip 50b, and further improve the stability and accuracy of detection. The whole closed-loop circuit is equipped with a transmission assembly 42ta for the yarn 50a or the fabric strip 50b to be circularly transmitted at a constant speed on the circuit. The detection device of the disclosure can detect the dynamic hairiness of the yarn 50a or the fabric strip 50b, and can predict various properties of the yarn 50a or the fabric strip 50b through the dynamic hairiness data. For example, one yarn or one fabric strip circulates a set of total hairiness data corresponding to this length. By comparing the hairiness data of each cycle, the friction and fuzziness of the yarn or the fabric strip can be obtained, and then the processing and weaving performance of the yarn and the performance of the fabric strip in the whole lifecycle from production and processing to use breakage can be predicted.

Specifically, when detecting the yarn, a detection device of a first embodiment as shown in FIGS. 1 and 2 is adopted. The tension adjustment unit 30 includes a slide rail 31a and a tension rod 32a arranged in the slide rail 31a. The yarn 50a is guided to the friction unit 40 through a lower side of the tension rod 32a, and the slide rail 31a is perpendicular to a transmission direction of the yarn 50a at the lower side of the tension rod 32a, which is used to automatically adjust the tension of the yarn 50a in real time by using the gravity of the tension rod 32a. The weight of the tension rod 32a is in the range of 2-20 grams (g). It should not be too heavy, which may cause excessive tension and affect friction and transmission; nor too light, which may make it difficult to achieve stable downward tension adjustment. In particular, the slide rail 31a is provided with a displacement scale line, and a length elongation of the yarn 50a is calculated by a displacement value of the tension rod 32a, so as to obtain the dynamic creep performance data of the yarn. The tension rod 32a may also be designed as a rod-shaped weight.

The hairiness detection unit 10 includes a hairiness acquisition module 11a, a displacement sensor lids, a data processing module 11dp, and guide rollers respectively arranged on front and rear guide paths of the hairiness acquisition module 11a, such as a first guide roller 12a, a second guide roller 13a, a first roller 14a (the first roller 14a provides a power source for yarn transmission) and a second roller 15a as shown in FIG. 1. The yarn 50a is fed into the entrance of the hairiness acquisition module 11a through the first guide roller 12a and the second guide roller 13a, and then is led out from the exit through the first roller 14a and the second roller 15a. The data processing module is used to convert hairiness data collected by the hairiness acquisition module into the number of hairiness per unit length of yarn, and classify the number of hairiness according to the hairiness length, for example, providing data on hairiness with lengths of 1 millimeter (mm), 3 mm, 5 mm, and 10 mm respectively. Each loop cycle produces a set of data on the hairiness of the yarn. Finally, through curve fitting, the abrasive resistance, creep, and other performance data of the yarn 50a are obtained. Through the detection device of the first embodiment of the disclosure, multiple sets of data can be matched in one-to-one correspondence, so as to comprehensively evaluate the yarn performance. For example, by comparing the hairiness data and creep data under different cycle times, the comprehensive influence of friction on yarn hairiness and creep can be judged. Different yarn hairiness data may be similar, but the creep differences can be significant, or the hairiness differences can be significant while the creep remains similar. Through the comprehensive evaluation of multiple sets of data, the yarn performance can be evaluated and predicted more comprehensively and reasonably. In an embodiment, a directional blowing mechanism 11db is arranged on one side of the hairiness detection unit 10 for directionally stretching the surface hairiness of the yarn, so that the fully stretched shape of the yarn hairiness is accurately projected on the hairiness acquisition module 11a.

In an embodiment, each of the hairiness acquisition module 11a and the data processing module is embodied by software stored in at least one memory and executable by at least one processor.

In particular, the loop circuit of the embodiment of the disclosure further includes an image acquisition assembly 16a for acquiring an appearance image of yarn evenness. In a specific embodiment, the image acquisition device 16a is arranged on a transmission path in front of the hairiness detection unit 10, such as a high-speed photographing device.

The tension adjustment unit 30 is arranged between the first guide unit 20 and the friction unit 40. The first guide unit 20 includes a first support rod 21 and multiple third guide rollers 22a arranged on the first support rod 21 for adjusting the guide of the yarn 50a. After the yarn 50a is led out from the first roller 14a and the second roller 15a, the yarn 50a is transmitted to the friction unit 40 through the third guide roller 22a at the lower side of the first support rod 21 and the tension rod 32a. The yarn 50a led out from the friction unit 40 is transmitted to the third guide roller 22a at the upper side of the first support rod 21, and then fed to the first guide roller 12a of the hairiness detection unit 10, so as to realize the closed-loop transmission of the yarn 50a. On the one hand, the first guide unit 20 serves to guide and transmit the yarn, and on the other hand, it is designed to address the issue where the yarn may be hindered in transmission due to the influence of its own weight and significant creep when the loop circuit is too long. By arranging the first guide unit 20 on the transmission path in front of the tension adjustment unit 30, the yarn path in the tension adjusting section is reduced, and the sensitivity and accuracy of free-falling tension adjustment are further improved. The distance between the tension adjustment unit 30, the first guide unit 20 and the friction unit 40 is 10-50 centimeters (cm), specifically, 15-30 cm; and the total length of the yarn loop is 10 meters (m).

The friction unit 40 includes a friction roller 42a, guide rollers (such as fourth guide rollers 43a in FIG. 1), and a transmission assembly 42ta for providing power to the friction roller 42a and adjusting rotation speed, so that the closed-loop yarn 50a is circulated and frictionally transmitted among the hairiness detection unit 10, the first guide unit 20, the tension adjustment unit 30 and the friction unit 40. The friction roller 42a and the fourth guide rollers 43a are arranged on a second support rod 41, and the rubbed yarn 50a is conveyed upward and then guided to the third guide roller 22a at the upper side of the first support rod 21. Through multiple groups of vertically upward guide rollers, it is convenient to deal with the transmission stability of longer yarns, reduce the path length in the horizontal direction, and save the occupied space of the detection device.

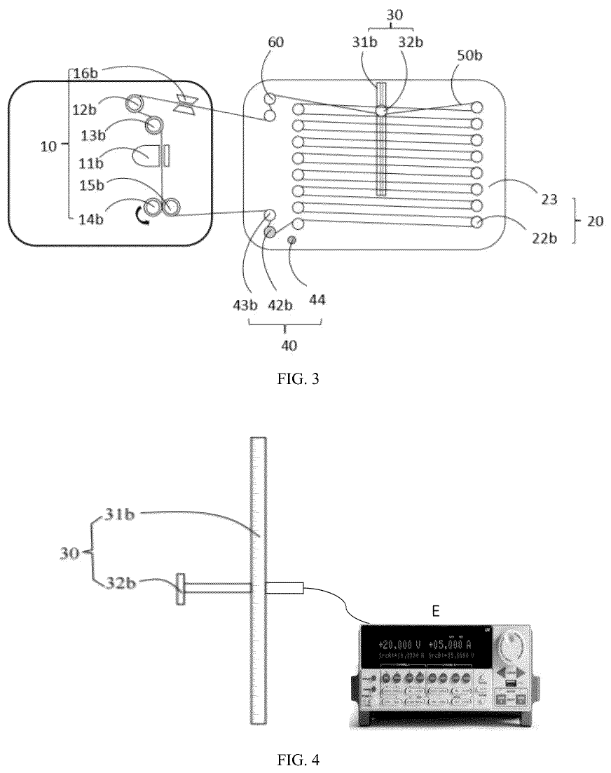

When detecting the fabric, the detection device of a second embodiment as shown in FIGS. 3 and 4 is adopted. The structure and realized functions (i.e., the relevant data parameters of the fabric strip 50b that can be detected) of the hairiness detection unit 10 are generally the same as those in the first embodiment, and will not be repeated here. It should be noted that, for the convenience of understanding, the reference signs of components of the hairiness detection unit 10 in the second embodiment are all denoted by b. For example, in FIG. 3, 11b represents the hairiness acquisition module, 12b represents the first guide roller, 13b represents the second guide roller, 14b represents the first roller, 15b represents the second roller, and 16b represents the image acquisition assembly.

The tension adjustment unit 30 is a free-falling tension adjusting electrostatic detection unit located directly behind the first guide unit 20 and is used to adjust and control the tension of the fabric strip 50b in the circulation process in real time and detect frictional electrostatic charges. The tension adjustment unit 30 includes a slide rail 31b and a tension rod 32b arranged on the slide rail 31b. The fabric strip 50b introduced by the first guide unit 20 enters the hairiness detection unit 10 through a lower part of the tension rod 32b. A movement direction of the tension rod 32b and a movement direction of the fabric strip 50b are perpendicular to each other. The weight of the tension rod 32b of the tension adjustment unit 30 is correspondingly set according to the width and thickness of the fabric strip 50b, and the greater the width and thickness of the fabric strip 50b, the higher the weight of the tension rod 32b. There is a certain distance between the tension adjustment unit 30 and the first guide unit 20, so that the transmission of the fabric strip 50b is not affected. The weight of the tension rod 32b should be in the range of 2-20 g, neither too heavy nor too light. If the weight of the tension rod 32b is too heavy, it will cause excessive tension, unexpected deformation, elongation, and wrinkles in the fabric strip 50b, thereby affecting the uniformity of friction and the accurate projection measurement of hairiness on the surface of the fabric strip 50b. On the other hand, if the weight of the tension rod 32b is too small, it will not be able to reduce the large fluctuations in tension of the fabric strip 50b caused by friction, making it difficult to achieve stable downward tension adjustment.

In some embodiments, the slide rail 31b is provided with a displacement scale line for obtaining dynamic creep data of the fabric strip 50b. Specifically, during cyclic friction process, the length of the fabric strip 50b will gradually increase due to creep (especially for elastic fabric strips). At this time, the tension rod 32b automatically adjusts the tension of the fabric strip 50b in real time by its own gravity, ensuring that the fabric strip 50b is always transmitted to the hairiness detection unit 10 with a constant tension, so as to ensure the stability of friction and transmission of the fabric strip 50b, and further improve the stability and accuracy of detection. The displacement sensor collects displacement scale on the slide rail 31b to calculate the dynamic creep elongation of the fabric strip 50b.

The first guide unit 20 includes multiple third guide rollers 22b and a fixing plate 23 for mounting the multiple third guide rollers 22b. As shown in FIG. 3, the multiple third guide rollers 22b are arranged on the fixing plate 23 in the form of 2×N, where N represents the number of rows of the third guide rollers 22b and 2 represents the number of third guide rollers 22b arranged in each row. In this way, the path length in the horizontal direction is reduced, and a longer fabric strip 50b can be continuously circulated in a relatively tight space, which saves the occupied space of the detection device and makes the measured result more representative. Specifically, after the fabric strip 50b is led out from the friction unit 40, it sequentially passes through two third guide rollers 22b in each row from bottom to top, and then is led into the tension adjustment unit 30. The tension adjustment unit 30 is mounted on the fixing plate 23.

In particular, a fabric material with static electricity after rubbing against the fabric strip 50b is arranged in the fabric guide groove of the third guide roller 22b, and the fabric material is wrapped on the surface of the fabric guide groove of the third guide roller 22b, ensuring that after repeated rubbing, the friction surface of the tested fabric strip 50b acquires electrostatic charges. In an embodiment, an electrometer E (shown in FIG. 4) is connected externally to the tension rod 32b to continuously measure the changes in surface charge (i.e., detecting frictional electrostatic charges) of the fabric strip 50b after repeated rubbing, reflecting the frictional power generation capability between different fabric materials.

The friction unit 40 includes a friction roller 42b, a speed control switch 44, and a transmission assembly 42ta. The transmission assembly 42ta is used to provide power for the friction roller 42b, so that the friction roller 42b rotates continuously to repeatedly rub the fabric strip 50b. The speed control switch 44 is used to adjust the rotation speed of the friction roller 42b, and different rotation speeds result in different frictional forces applied to the fabric strip 50b. With the increase of rotation speed, the friction force on the fabric strip 50b increases continuously. In the actual testing process, the rotation speed of the friction roller 42b can be adjusted freely to regulate the friction force applied to the fabric strip 50b.

The friction unit 40 further includes a fourth guide roller 43b arranged at an entrance end of the fabric strip 50b of the friction roller 42b, and the fabric strip 50b output from the hairiness detection unit 10 is guided onto the friction roller 42b by the fourth guide roller 43b, and a fabric surface of the fabric strip 50b is closely attached to the friction roller 42b.

The device for whole lifecycle multi-indicator synchronous detection of yarns or fabrics in this embodiment further includes a second guide unit 60 arranged between the tension adjustment unit 30 and the hairiness detecting unit 10, and the second guide unit 60 is installed on the fixing plate 23. The second guide unit 60 includes two vertically arranged fifth guide rollers, the fabric strip 50b is guided from the tension adjustment unit 30 to the upper fifth guide roller, and then is guided out from the lower fifth guide roller and enters the hairiness detection unit 10.

It should be understood by those skilled in the art that the device for whole lifecycle multi-indicator synchronous detection of yarns or fabrics provided in the first embodiment and the second embodiment can realize the detection of yarns or fabric strips.

The disclosure also provides a method for whole lifecycle multi-indicator synchronous detection of yarns or fabrics, which adopts the device for whole lifecycle multi-indicator synchronous detection of yarns or fabrics, and includes the following steps:
S1, a closed-loop yarn with a preset length or a fabric strip subjected to protective pretreatment is sequentially passed through the hairiness detection unit 10, the first guide unit 20, the tension adjustment unit 30 and the friction unit 40 to form a circulating transmission loop;
S2, the hairiness detection unit and a transmission assembly are started to drive the yarn or the fabric strip at a uniform speed, and a hairiness length and a quantity on the yarn or the fabric strip are acquired in real time until the yarn or the fabric strip is worn and broken; and
S3, all broken data are processed to obtain multiple groups of performance data of the yarn or the fabric strip, so as to complete the whole lifecycle multi-indicator synchronous detection of the yarn or the fabric strip.

Specifically, the preset length of the closed-loop yarn or the fabric strip is 10 m. The transmission speed of the yarn or fabric strip is 5-100 meter per minute (m/min), and the transmission speed of the yarn or the fabric strip is 30 m/min according to the test standard of yarn hairiness. The friction force of the friction unit 40 is determined by the surface roughness of the yarn or the fabric strip, the roughness of the abrasive paper coated on the friction unit 40, and the weight of the tension rod 32a or the tension rod 32b that can automatically adjust the tension. The greater the roughness and friction of the abrasive paper coated on the friction unit 40, the heavier the tension rod 32a or the tension rod 32b and the greater the friction force.

In addition, for fabric strips 50b such as knitted fabrics and woven fabrics that are prone to edge fraying or deformation, such as woolen fabrics and non-woven fabrics, the detection results will be affected or even the circulation process will be terminated because of the scattered or deformed edges. Therefore, it is necessary to carry out protective pretreatment for such fabric strips 50b in advance. As shown in FIG. 5, the protective pretreatment is specifically as follows. Firstly, the fabric strip 50b is spread flat, and a double-sided adhesive tape 70 is then affixed in a middle of the fabric strip 50b in the width direction. Next, two side edges of the fabric strip 50b are folded towards the middle, allowing the double-sided adhesive tape 70 to bond the two sides of the fabric strip 50b. Finally, a layer of double-sided adhesive tape 70 is applied on top of a bonded area of the fabric strip 50b (the release paper on the outside of this layer of double-sided adhesive tape 70 is retained), resulting in a single-sided structured fabric strip 50b, with one side being the fabric strip 50b and the other side being a protective layer of double-sided adhesive tape 70. The fabric side of the single-sided structured fabric strip 50b is closely adhered to the friction unit 40. In this way, not only is the edge of the fabric strip 50b bonded with double-sided adhesive tape 70 to prevent the edge from being scattered or deformed, but also the double-sided adhesive tape 70 serves as a skeleton, providing certain support to the fabric strip 50b. For the elastic fabric strip 50b, reinforcement can be achieved by using elastic double-sided adhesive tape 70 that matches the elasticity of the fabric strip 50b. Additionally, the width of the double-sided adhesive tape 70 is half the width of the fabric strip 50b.

Multiple sets of performance data of yarn or fabric strip include real-time hairiness data, cyclic abrasion resistance data, pilling data, dynamic creep data, weight loss data from friction of yarn or fabric strip, evenness appearance data from friction of yarn or fabric strip, predicted yarn weaving performance and frictional electrostatic charge data of fabric strip. Specifically, the number of cycles at which the yarn or fabric strip breaks can be used to characterize the cyclic abrasion resistance of the yarn or fabric strip. By using the hairiness detection unit 10 or the image acquisition assembly 16a or 16b, the amount of pilling can be obtained. Through the real-time displacement data of the tension rod 32a or 32b, the dynamic creep performance can be obtained, and the total creep length of the yarn or fabric strip can be obtained by comparing the broken length of the yarn or the rubbed length of the fabric strip with the initial length. The weight loss data from friction of yarn or fabric strip is obtained by comparing the broken or rubbed weight with the initial weight. The evenness appearance data from friction of yarn or fabric strip is obtained by image acquisition assembly 16a or 16b. The frictional electrostatic charge data is obtained by the external electrometer at the tension rod 32b. The weaving performance of yarn or the performance of fabric strip can be comprehensively predicted by the performance data obtained above. For example, the yarn or fabric strip will be subjected to certain repeated friction force during weaving. By setting the friction force of the disclosure equal to that of the actual weaving process, the dynamic hairiness and creep data changes are tested, and the performance of the weaving process or the fabric strip is simulated and predicted. According to the disclosure, the influence of sizing process on the comprehensive performance of yarn weaving is predicted by comparing the dynamic hairiness and abrasion resistance detection results of sized and unsized yarn or fabric strip.

The performance of yarns with different thicknesses is characterized as follows. Specifically, yarns with yarn counts of 18, 24, 36 and 48 tex are spun by cotton roving respectively, and the closed-loop yarn of 10 m above is subjected to cyclic friction test by using the device for whole lifecycle multi-indicator synchronous detection of yarns or fabrics provided by the first embodiment of the disclosure, so as to dynamically monitor seven indicators including the number change of hairiness, the shape change of evenness during the yarn friction process, the yarn pilling after friction, the yarn creep during friction, the yarn weight loss before and after friction, the yarn friction cycle times, and prediction of yarn weaving performance.

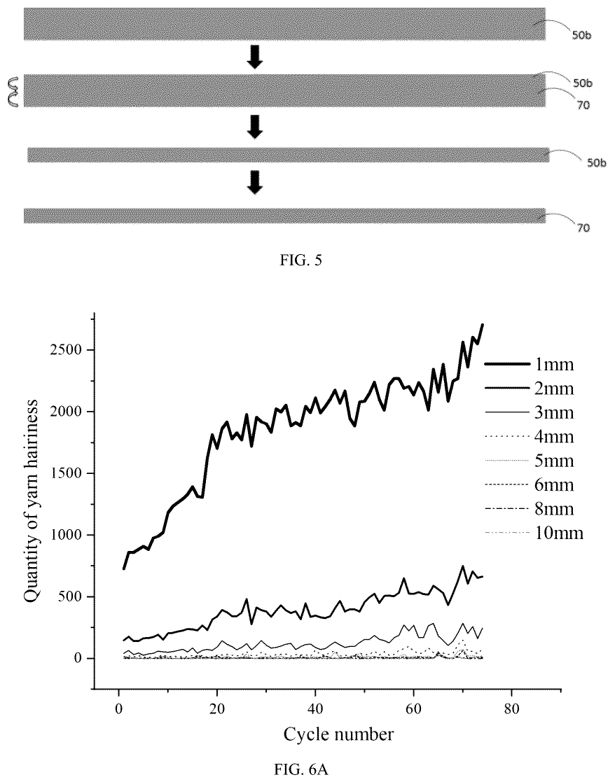

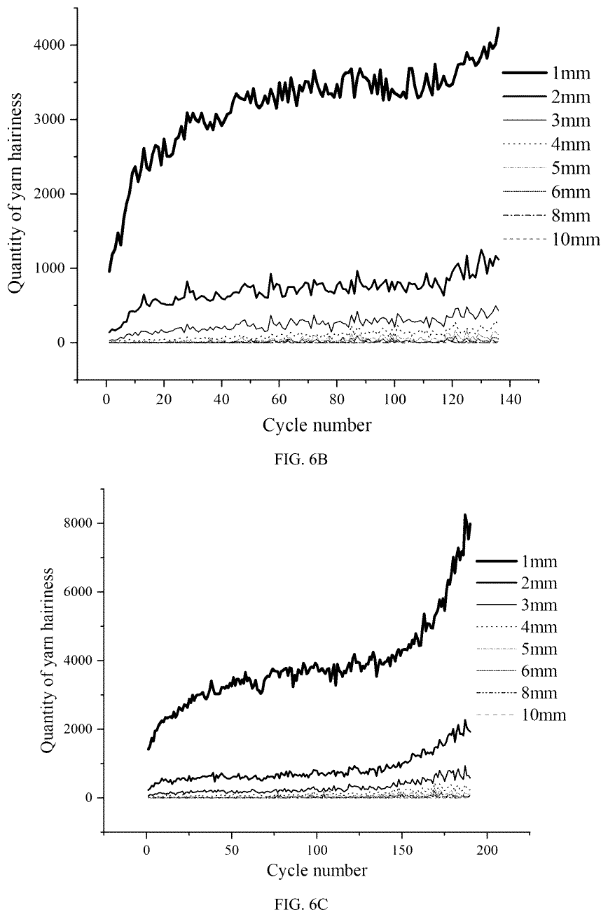

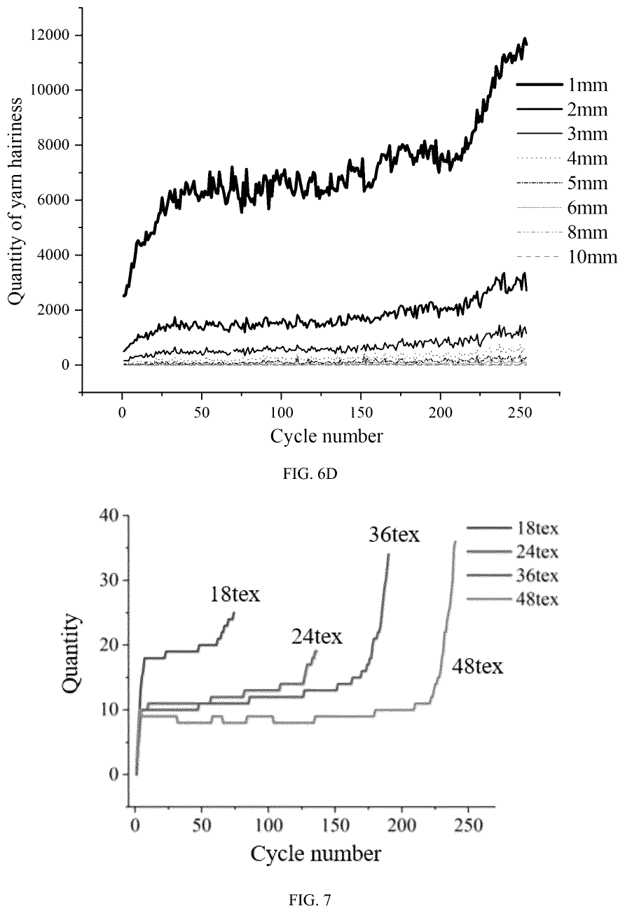

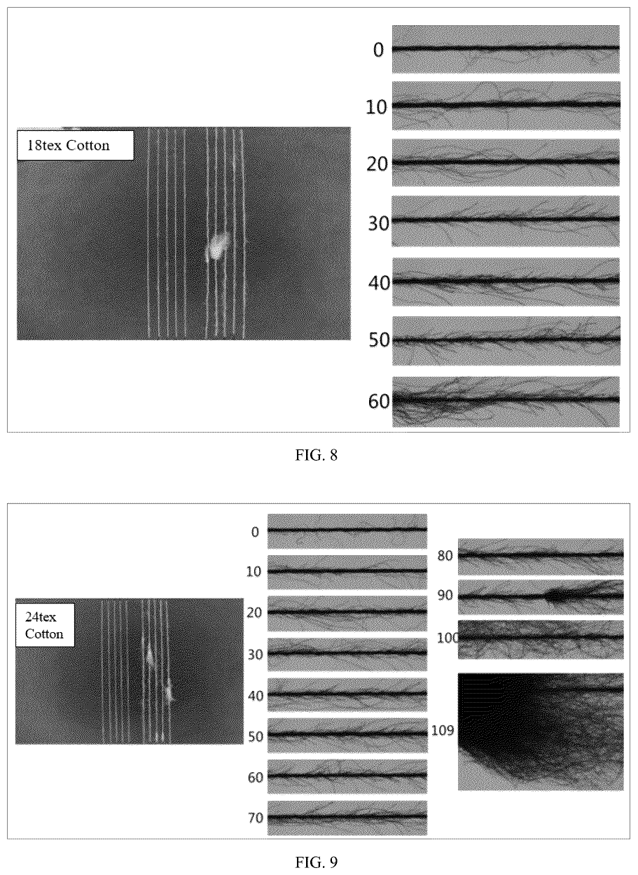

As shown in FIGS. 6A-6D, it is obvious that the larger the yarn diameter, the more cycles it can withstand before breaking, indicating better abrasion resistance. The number of cycles for yarn breakage at 18, 24, 36, and 48 tex yarn increases successively to 74, 136, 190, and 240 times, respectively, accompanied by a gradual increase in hairiness. Notably, when the number of cycles for 36 tex yarn exceeds 150 times, the number of hairiness increases sharply, rising from 4000 to 8000. This suggests that as the diameter of cotton yarn increases, its strength also increases, but the number of hairiness simultaneously multiplies, and friction more readily generates hairiness.

TABLE 1
Test results of yarn dynamic creep length and weight loss

| Yarn count (tex) | Dynamic creep length (cm) | Weight loss (g) |
|---|---|---|
| 18 | 12.0 | 0.0257 |
| 24 | 31.5 | 0.0361 |
| 36 | 82.0 | 0.1164 |
| 48 | 147.5 | 0.1213 |

As can be seen from Table 1, the creep elongation and mass loss of yarn at break increase with the increase of yarn fineness.

In conclusion, the device and the method for whole lifecycle multi-indicator synchronous detection of yarns or fabrics provided by the disclosure ensure that the yarn or fabric strip always maintains a certain tension in the process of friction and creep by setting up the closed-loop friction circuit and the free-falling tension adjustment unit on the circuit, thus improving the friction stability. At the same time, real-time data such as hairiness change of the yarn or fabric, yarn imperfections, abrasion resistance times, length change of the yarn or fabric are collected until the yarn or fabric is worn and broken, and then the abrasion resistance, strength, creep and other properties of different materials, structures, thicknesses and functional yarns or fabric strips are evaluated and measured, so that the rapid detection of multiple performance indicators of functional yarns or fabric strips can be realized, and the practicability and convenience are significantly improved. The disclosure provides a novel device and detection idea for the comprehensive performance evaluation of yarns or fabric strips, and is expected to obtain a new performance evaluation standard of yarns or fabric strips, thereby improving the detection efficiency and accuracy of the performance of yarns or fabric strips.

The above embodiments are only used to illustrate the technical scheme of the disclosure, but not to limit it. Although the disclosure has been described in detail with reference to the illustrated embodiments, it should be understood by those skilled in the art that the technical scheme of the disclosure can be modified or replaced by equivalents without departing from the spirit and scope of the technical scheme of the disclosure.

What is claimed is:

1. A device for whole lifecycle multi-indicator synchronous detection of a yarn or a fabric, comprising: a hairiness detection unit, a first guide unit, a tension adjustment unit and a friction unit;
wherein the device is configured to make the yarn or the fabric strip with a preset length be circularly rubbed on the friction unit, and the hairiness detection unit is configured to collect hairiness data of the yarn or the fabric strip in real time; and the tension adjustment unit is a free-falling tension adjustment unit and is configured to automatically adjust a tension of the yarn or the fabric strip in real time to ensure stability of friction and transmission of the yarn or the fabric strip.

2. The device as claimed in claim 1, wherein the tension adjustment unit comprises a slide rail and a tension rod arranged in the slide rail; the slide rail is perpendicular to a transmission direction of the yarn or the fabric strip, and the slide rail is configured to automatically adjust the tension of the yarn or the fabric strip in real time by using a gravity of the tension rod.

3. The device as claimed in claim 2, wherein the tension rod is externally connected to an electrometer, and the electrometer is configured to real-time detect frictional electrostatic charges of the yarn or the fabric strip in a cycle process.

4. The device as claimed in claim 2, wherein the slide rail is provided with a displacement scale line configured to obtain dynamic creep data of the yarn or the fabric strip.

5. The device as claimed in claim 1, wherein the hairiness detection unit comprises a hairiness acquisition module, a displacement sensor, a data processing module, and guide rollers respectively arranged on front and rear guide paths of the hairiness acquisition module;
the data processing module is configured to convert the hairiness data acquired by the hairiness acquisition module into a number of hairiness per unit length of the yarn or per unit area of the fabric strip, and classify the number of hairiness according to a hairiness length; and
a side of the hairiness detection unit is provided with a directional blowing mechanism, and the directional blowing mechanism is configured to directionally stretch hairiness of a surface of the yarn or the fabric strip to project a stretched shape of the hairiness of the yarn or the fabric strip on the hairiness acquisition module.

6. The device as claimed in claim 1, wherein the friction unit comprises a friction roller, guide rollers, and a transmission assembly configured provide power to the friction roller and adjust a rotation speed, to make the yarn or the fabric strip be capable of being circulated and frictionally transmitted among the hairiness detection unit, the first guide unit, the tension adjustment unit and the friction unit.

7. The device as claimed in claim 2, wherein the tension adjustment unit is arranged between the first guide unit and the friction unit, and the first guide unit comprises a first support rod and a plurality of guide rollers arranged on the first support rod configured to adjust a guide of the yarn.

8. The device as claimed in claim 2, wherein the tension adjustment unit is located behind the first guide unit, and the first guide unit comprises a plurality of guide rollers and a fixing plate configured to mount the guide rollers; the plurality of the guide rollers are arranged on the fixing plate in a form of 2×N, where N represents a number of rows of the plurality of guide rollers, and 2 represents a number of the guide rollers arranged in each row; and the tension adjustment unit is mounted on the fixing plate.

9. The device as claimed in claim 1, further comprising an image acquisition assembly configured to acquire an evenness appearance image of the yarn.

10. A method for whole lifecycle multi-indicator synchronous detection of a yarn or a fabric, adopting the device for whole lifecycle multi-indicator synchronous detection of the yarn or the fabric as claimed in claim 1, comprising the following steps:
S1, sequentially passing the yarn with the preset length or the fabric strip subjected to protective pretreatment through the hairiness detection unit, the first guide unit, the tension adjustment unit and the friction unit to form a circulating transmission loop;
S2, starting the hairiness detection unit and a transmission assembly, so as to drive the yarn or the fabric strip at a uniform speed, and acquiring a hairiness length and a quantity on the yarn or the fabric strip in real time until the yarn or the fabric strip is worn and broken; and
S3, processing all broken data to obtain a plurality of groups of performance data of the yarn or the fabric strip, so as to complete the whole lifecycle multi-indicator synchronous detection of the yarn or the fabric strip; and
wherein the plurality of groups of performance data of the yarn or the fabric strip include cyclic abrasion resistance data, pilling data, dynamic creep data, weight loss data of the yarn or the fabric strip from friction, evenness appearance data of the yarn or the fabric strip from friction, and predicted yarn weaving performance.

11. The method as claimed in claim 10, wherein the protective pretreatment of the fabric strip specifically comprises the following steps:
sticking a double-sided adhesive tape at a middle of the fabric strip in a width direction, and folding two sides of the fabric strip to the middle, to make the double-sided adhesive tape be bonded to edges of the two side of the fabric strip; then applying a layer of the double-sided adhesive on a top of a bonded area of the fabric strip, and retaining a release paper of the double-sided adhesive on the top.

12. A detection device, comprising: a hairiness detection unit, a first guide unit, a tension adjustment unit and a friction unit;
wherein the friction unit is configured to rub a to-be-detected object; the tension adjustment unit is configured to automatically adjust a tension of the to-be-detected object; the first guide unit is configured to guide the to-be-detected object; and the hairiness detection unit is configured to collect hairiness data of the to-be-detected object; and
wherein the to-be-detected object is sequentially passed through the hairiness detection unit, the first guide unit, the tension adjustment unit and the friction unit to form a loop, so as to obtain performance data to complete detection of the to-be-detected object.

13. The detection device as claimed in claim 12, wherein the tension adjustment unit comprises a slide rail and a tension rod arranged in the slide rail; the slide rail is perpendicular to a transmission direction of the to-be-detected object, and the slide rail is configured to automatically adjust the tension of the to-be-detected object in real time by using a gravity of the tension rod.

14. The detection device as claimed in claim 13, wherein the to-be-detected object is a yarn with a preset length.

15. The detection device as claimed in claim 13, wherein the to-be-detected object is a fabric strip.

16. The detection device as claimed in claim 15, wherein the hairiness detection unit comprises a hairiness acquisition module, a first guide roller, a second guide roller, a first roller, and a second roller;
wherein the yarn is fed into an entrance of the hairiness acquisition module through the first guide roller and the second guide roller, and then is led out from an exit through the first roller and the second roller; and
wherein the hairiness acquisition module is configured to collect hairiness data of the yarn.

17. The detection device as claimed in claim 16, further comprising an image acquisition assembly arranged on a transmission path of the yarn, wherein the image acquisition assembly is configured to acquiring an evenness appearance image of the yarn.

18. The detection device as claimed in claim 17, wherein the tension adjustment unit is arranged between the first guide unit and the friction unit;
wherein the first guide unit comprises a first support rod and a plurality of third guide rollers arranged on the first support rod configured to adjust a guide of the yarn;
wherein the friction unit comprises a friction roller and fourth guide rollers; and
wherein the yarn led out from the first roller and the second roller is transmitted to the friction roller of the friction unit through one third guide roller at a lower side of the first support rod; the yarn is frictionally transmitted by the friction roller of the friction unit and then conveyed upward through the fourth guide rollers to out from the friction unit; the yarn led out from the friction unit is transmitted to one third guide roller at an upper side of the first support rod, and then fed to the first guide roller of the hairiness detection unit, so as to realize a closed-loop transmission of the yarn.

19. The detection device as claimed in claim 15, wherein the first guide unit comprises a plurality of third guide rollers and a fixing plate configured to mount the plurality of third guide rollers; the plurality of the third guide rollers are arranged on the fixing plate in a form of 2×N, where N represents a number of rows of the plurality of third guide rollers, and 2 represents a number of the third guide rollers arranged in each row; and the tension adjustment unit is mounted on the fixing plate.

20. The detection device as claimed in claim 19, wherein the friction unit comprises a friction roller and a speed control switch; the friction roller is configured to be rotated continuously to repeatedly rub the fabric strip, and the speed control switch is configured to adjust a rotation speed of the friction roller.
